(19) United States
(12) Patent Application Publication
(10) Pub. No.: US 2012/0071543 A1
(43) Pub. Date: Mar. 22, 2012

(54) FORMULATIONS OF DILUTED GENETIC MATERIAL AND METHODS FOR MAKING SAME

(76) Inventors: Jacob L. CARTER, Pleasant Grove, UT (US); Matthew E. Gruwell, Erie, PA (US)

(21) Appl. No.: 13/107,663

(22) Filed: May 13, 2011

Related U.S. Application Data
(60) Provisional application No. 61/334,770, filed on May 14, 2010.
(60) Provisional application No. 61/358,725, filed on Jun. 25, 2010.

(51) Int. Cl. A61K 48/00; B65D 85/00

(52) U.S. Cl. 514/44 R; 206/459.5

(57) ABSTRACT

Formulations of highly diluted genetic material are disclosed. The genetic material may be human genetic material that is associated with a health effect in humans. For example, the genetic material may cause, ameliorate, reduce, or otherwise affect any form of illness, disease, and/or overall wellness in humans. The formulations are made by serially diluting an initial mixture that includes the genetic material. In one embodiment, a patient's genetic material is tested and the results are used to prepare a formulation specifically for the genetic predispositions and/or indicators exhibited by the patient. The formulations include homeopathic remedies.

### CROSS-REFERENCE TO RELATED PATENT APPLICATIONS

This patent application claims the benefit of U.S. Provisional Patent Application No. 61/334,770, titled “System and Method for the Generation of Genetically Matched Homeopathy,” filed on 14 May 2010, and U.S. Provisional Patent Application No. 61/358,725, titled “System and Method for the Generation of Genetically Matched Homeopathy,” filed on 25 Jun. 2010, the entire contents of both are incorporated by reference herein. In the event of a conflict, the subject matter explicitly recited or shown herein controls over any subject matter incorporated by reference. The incorporated subject matter should not be used to limit or narrow the scope of the explicitly recited or depicted subject matter.

### BACKGROUND

Many Americans use complementary and alternative medicine (CAM) in pursuit of health and well-being. The 2007 National Health Interview Survey (NHIS), which included a comprehensive survey of CAM use by Americans, showed that approximately 38 percent of adults use CAM.

CAM is a group of diverse medical and health care systems, practices, and products that are not generally considered part of conventional medicine. Conventional medicine (also called Western or allopathic medicine) is medicine as practiced by holders of M.D. and D.O. degrees and by allied health professionals, such as physical therapists, psychologists, and registered nurses. The boundaries between CAM and conventional medicine are not absolute and specific CAM practices may, over time, become widely accepted.

The term “complementary medicine” refers to use of CAM together with conventional medicine, such as using acupuncture in addition to conventional medicinal techniques used to help lessen pain. Most use of CAM by Americans is complementary. “Alternative medicine” refers to use of CAM in place of conventional medicine. “Integrative medicine” (also called integrated medicine) refers to a practice that combines both conventional and CAM treatments for which there is evidence of safety and effectiveness.

One type of CAM is the whole medical system of homeopathy. It is a complete system of theory and practice that has evolved over time in different cultures and apart from conventional medicine. Homeopathy is used for wellness and prevention and to treat many diseases and conditions.

Homeopathy is founded on principles established in pharmacology and biology. Homeopathy seeks to stimulate the body's ability to heal itself by giving very small doses of highly diluted substances. This therapeutic method was first developed in the 18^{th} century by a German physician Samuel Hahnemann.

Hahnemann articulated two of the foundational principles of homeopathy. The first is the principle or law of similars (or “like cures like”). This principle states that a disease can be cured by a substance that produces similar symptoms in healthy people. This idea, which can be traced back to Hippocrates, was further developed by Hahnemann after he repeatedly ingested cinchona bark, a popular treatment for malaria, and found that he developed the symptoms of the disease. Hahnemann theorized that if a substance could cause disease symptoms in a healthy person, small amounts could cure a sick person who had similar symptoms. The second principle articulated by Hahnemann is the principle of dilutions (or “law of minimum dose”). This principle states that the lower the dose of the medication, the greater its effectiveness.

In 1877, Hugo Schultz postulated that the effect of a stimulus on a living cell is indirect and proportional to its intensity and quantity. Later, in 1888, Schultz demonstrated that very low concentrations of yeast toxins increased yeast growth over 100 fold. Concurrently, the psychiatrist Rudolph Arndt developed his “basic law of biology,” which states that weak stimuli slightly accelerate the vital activity, middle-strong stimuli raise it, strong stimuli suppresses it, and very strong stimuli halt vital activity.

These separate observations were formulated by Arndt in 1888 into one of the earliest laws of pharmacology representing the homeopathic effect, the Arndt-Schultz law, which states: every stimulus on a living cell elicits an activity, which is inversely proportional to the intensity of the stimulus (Martius F. Das Arndt-Schultz Gnindgesetz, Muench Med. Wschr., 1923, 70(31):1005-1006). This law was later restated by Hueppe as: for every substance, small doses stimulate, moderate doses inhibit, large doses kill.

One of the basic tenets of homeopathy is that a cure or treatment for a disease can be evoked by using a high dilution of a material that resembles but is different from the cause of the disease. Homeopathy is widely accepted as a useful therapeutic and has been demonstrated to have characteristic and reproducible effects. A critical review of more than 100 controlled and/or clinical studies of homeopathy determined that patients received positive healing benefits from homeopathy beyond the placebo effect (Kleijnen, J. et al. 1991 Brit. Med. J. 302:316-323; Linde, K., Clausius, N., Ramirez, G., Melchart, D., Eitel, F., Hedges, L. V., Jonas, W. B., 1997, Lancet, 350:834-843; Reilly, D., et al, 1994, Lancet, 344:1601-1608).

Many homeopathic remedies are used in very low concentrations on the order of micrograms (10⁻⁶ M) and nanograms (10⁻¹² M); however, in other homeopathic preparations, the dilutions exceed Avogadro's number (6.023×10⁻²³). When homeopathic compounds are repetitively diluted 1:10 (written as “X”) or 1:100 (written as “C”), with repeated succussions (similar to vortexing) at least 24 times, a potency is achieved (10⁻²⁴ or 24X or 12C) that is so highly dilute that the probability of a single molecule of the original substance remaining in the volume used is less than 1×10⁻¹⁰.

Homeopathic practitioners believe that the potency of a compound increases with increasing dilutions. In traditional homeopathic practice, the standard homeopathic dosage is 10-15 drops of a 10⁻¹² molar, or 6C, solution administered two to three times per day. A 10⁻⁶⁰ molar or 30C may be given one to three times per day. A 10⁻⁴⁰⁰ molar or 200C may be given only one time per month or year. A 6C dilution approximates 1 picogram/ml, which is used in cell culture but would be considered a lower than physiological dose when administered to a patient either orally, topically or by injection.

Highly dilute homeopathic remedies have been effective in treating some conditions, including viral infections, in vivo. Homeopathic dilutions of 200× to 1000× of typhoidinum, hydrophobinum, tuberculinum, nux vomica and malandrinum 100% inhibited pock-like lesions caused by a chicken embryo DNA virus on the chorio-allantoic membrane compared to controls (Singh, L. M. and Gupta, G. 1985 Brit. Homeopathy 74:168-174). Other homeopathic remedies, the same active compound at different homeopathic concentrations, or control phosphate buffered solution (PBS), had lesser or no effect.

While the exact mechanism of action of homeopathic remedies is unknown, magnetic resonance image measurements on serial dilutions of substances indicate that the hydroxyl (OH) groups in the solvent of solutions continue to change as dilutions become successively higher (Sacks, A. D. 1983 J. Holistic Med. 5:175-176; Smith, R. and Boericke, G. 1968 J. Am. Inst. Homeopathy 61:197-212; Smith, R. and Boericke, G. 1966 J. Am. Inst. Homeopathy 59:263-279). It is clear that the specific effects of homeopathics are of a non-molecular origin, yet provide potent biological activities that are clinically effective.

It has been postulated that highly dilute compounds transfer biological activity to cells by electromagnetic fields (Benveniste, J. 1993 Frontier Perspectives 3:13-15). Del Giudice et al. have hypothesized that interactions between the electric dipoles of water and the radiation fields of a charged molecule generate a permanent polarization of water which becomes coherent and has the ability to transmit specific information to cell receptors, somewhat like a laser (Del Giudice, E., Preparata, G., Vitiello, G. 1988, Phys. Rev. Lett. 61:1085-1088).

Although homeopathy has proven to be effective, there are no current homeopathic remedies that take advantage of genetic influences on human health. The following disclosure is directed to genetics based homeopathic remedies used to influence human health including treating diseases and conditions and improving overall wellness.

### SUMMARY

A highly diluted formulation and methods for making and using the same are disclosed herein. The formulation is prepared using genetic material that is associated with a health effect in humans. For example, the genetic material may represent an increased susceptibility or resistance to a specific disease or adverse health condition. The genetic material may also be associated with positive health effects such as increased muscle development, athletic endurance, fast twitch muscle response, and anti-aging.

Although the formulation is prepared primarily for use by humans, it should be appreciated that it may also be made to influence the health of animals. In the case of animals, the included genetic material is associated with one or more health effects in the species, or specific animal, for which the formulation is made. Formulations may be made for animals such as cats, dogs, horses, and so forth.

In one embodiment, the genetic material includes one or more alleles of a genomic loci that are associated with various health effects in humans. A mixture that includes the one or more alleles of defined genomic lici are prepared by identifying DNA that contains the alleles and amplifying the alleles in the DNA to produce a sufficient quantity to make the formulation. The alleles may be amplified using any suitable technique such as locus-specific PCR or carrier organism replication. The specific nucleotide strands are purified and kept separate or mixed with other key loci to produce the mixture of genetic material.

The genetic material may be diluted to produce the formulation in any of a number of ways. In general, the genetic material is mixed with a diluting agent to produce a first mixture. The diluting agent may be water, ethanol, glycerin, lactose, and/or sucrose, among other materials. The first mixture is serially diluted to produce the final formulation. Each increasingly dilute mixture is succussed or vigorously shaken to potentize or activate it. In one embodiment, the final formulation is a homeopathic remedy.

Any suitable dilution ratio may be used to dilute the first mixture. The dilution ratio for each successive, increasingly dilute mixture may be the same or different. In one embodiment, the first mixture is diluted on a decimal or centesimal scale using the same dilution ratio for each step.

The increasingly dilute mixtures may be succussed in a number of different ways. At a minimum, the succussion process will include vigorous shaking However, succussion may also include subjecting the mixtures to an impact force. It may also include vortexing the mixtures, which is a specific type of vigorous shaking The mixtures may be succussed for any suitable length of time and with or without a pause between each vigorous shaking episode.

The formulation includes pre-manufactured standard formulations that are prepared from genetic material that is widely associated with different health effects. The formulations may also be custom made and tailored to match the genetic profile of an individual patient, family, population, or haplogroup.

The formulation may be provided in a variety of forms. Examples of common forms include liquid dilutions that are dispensed from a dropper, pellets, tablets, and capsules. Other forms include ointments, gels, and suppositories. The formulations may be sold over the counter or by prescription.

In one embodiment, the formulation is made by obtaining genetic material from a patient and analyzing the genetic material to identify genetic material that indicates the patient is susceptible or predisposed to a disease or some other health effect. The genetic material is isolated and amplified and used as the starting material to prepare a highly dilute formulation. The formulation is administered to the patient to treat or otherwise address diseases that the patient is susceptible to and to increase the patient's overall wellness. The formulation may be a homeopathic remedy.

According to one embodiment, the patient is genetically mapped and a homeopathic remedy is prepared to treat or otherwise support the body's ability to overcome any illness, disease, or other health effect that the patient is genetically predisposed towards.

The homeopathic remedy is provided to naturally support and nurture the body's ability to overcome or manage undesirable health conditions and promote the body's ability to enhance desirable health effects. The remedy supports the body's own genetic processes that are the source of many individual health effects. In particular, the remedy may support the patient's genetic health or relieve the symptoms associated with genetic conditions.

The term “genetic material” means any polynucleotide, nucleotide sequence, gene, pseudogene, part of a gene or pseudogene, group of genes and/or pseudogenes, DNA or RNA molecule, fragment of DNA or RNA, group of DNA and/or RNA molecules, mobile genetic element, transposon (Class I or II), promoter region, open reading frame, or the entire genome of an organism. The term “native genetic material” refers to genetic material that is part of a natural cellular environment.

The term “extracted genetic material” means genetic material removed from the natural cellular environment. The term “synthesized genetic material” means genetic material that has not been removed from the natural cellular environment such as genetic material synthesized chemically or heterologously. Synthesized genetic material includes, for example, recombinant DNA, complementary DNA, polymerase chain reactor (PCR) product, reverse transcriptase PCR product, and the like.

The term “non-native genetic material” refers to genetic material that is not part of a natural cellular environment and includes extracted genetic material and synthesized genetic material. The term “isolated genetic material” refers to extracted or synthesized genetic material that exists substantially separate from other cellular components normally associated with native genetic material including proteins and other nucleotide sequences that are part of the remainder of the genome. The term “human genetic material” refers to genetic material found in or associated with humans and can be extracted genetic material or synthesized genetic material.

The term “v/v” means the volume fraction of a material (i.e., the volume of the specified material divided by the volume of the total solution). The term “w/w” means the weight fraction of a material (i.e., the weight of the specified material divided by the weight of the total solution). The two terms may expressed as a percent by multiplying the value by 100.

The Summary is provided to introduce a selection of concepts in a simplified form that are further described below in the Detailed Description. The Summary and the Background are not intended to identify key concepts or essential aspects of the disclosed subject matter, nor should they be used to constrict or limit the scope of the claims. For example, the scope of the claims should not be limited based on whether the recited subject matter includes any or all aspects noted in the Summary and/or addresses any of the issues noted in the Background.

### DETAILED DESCRIPTION

A formulation that includes highly diluted genetic material may be prepared and administered to humans or animals to treat or otherwise address health effects that have a genetic component. The formulation is created by mixing the genetic material with a diluting agent and serially diluting the resulting mixture. The final formulation can be a homeopathic remedy that is administered and dispensed in a similar way to other homeopathic remedies.

The genetic material is used as the source material to prepare the formulation. The genetic material is typically selected because it is associated with a health effect in the patient such as an increased susceptibility or resistance to an illness, disease, ailment, malady, birth defect, genetic condition, inherited genetic risk factor, or, on the other hand, a beneficial health characteristic such as improved vision, better fast muscle speed, weight loss, metabolism speed, hair growth, and the like. The genetic material may be selected for other reasons beyond being associated with a health effect.

The formulation may be a standard off the shelf formulation that is produced from genetic material associated with common diseases, traits, or other health effects. In an alternative embodiment, the formulation may be custom made based on a patient's genetic profile. A genetic sample from the patient (e.g., blood, hair, skin, amniotic fluid, buccal smear, etc.) may be analyzed to identify a genetic predisposition towards certain health effects. The identified genetic material is then isolated, amplified and purified to produce a sufficient amount of the genetic material to make the formulation.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

Many health effects in humans are caused or influenced by a person's genes. Genes are the basic units of heredity. They contain sequences of DNA (deoxyribonucleic acid) which produce proteins that control cells and enable our body to develop and function. Genes are located at specific positions on chromosomes inside each cell nucleus.

Humans have 46 chromosomes organized into a set of 23 pairs—one in each pair inherited from each parent. Each pair carries numerous genes responsible for different physical characteristics. These characteristics are known as traits or phenotypes, and a single trait can be controlled by a number of genes located on different sections of different chromosomes. 22 pairs of chromosomes are responsible for non-sex characteristics and are called autosomes. The remaining pair are sex chromosomes and they determine gender. Males (XY) inherit a Y chromosome from their father and an X from their mother. Females (XX) inherit an X from each parent.

The human genome is all the genetic information contained in the DNA of a set of chromosomes from a cell nucleus. An individual's unique, individual genetic makeup is called a genotype which we inherit from our parents through sexual reproduction. Our phenotype is the physical expression of our genotype. Phenotype can also be influenced by environment and random genetic changes.

A genetic mutation is a change in DNA and is often random. Many are not necessarily harmful and are responsible for a wide range of genetic variations in humans. Some mutations can be passed down the generations and underlie the process of evolution. When a genetic mutation alters the function of some cells, it can cause or make a person more susceptible or predisposed toward certain diseases, physical defects, and/or other health effects. A person is said to be affected by a genetic condition if they express the phenotype for a faulty genotype—i.e. they have a disease or condition arising from a genetic abnormality.

A simple overview of genetic mutations and the conditions they may cause is given below. All cells are the product of the division of pre-existing cells (through the processes of mitosis and meiosis). The division and replication of genetic material during cell division are crucial factors for producing normal offspring. Somatic mutations (sometimes called sporadic or acquired mutations) occur in non-sex cells during a person's lifetime and can lead to conditions which are not usually hereditary but still genetic in nature. In contrast, germline mutations occur in the sex cells that lead to embryo formation and can be passed on to subsequent offspring. Studying patterns of inheritance of a condition or disease in a family, by examining a family tree or pedigree, can identify the mode of transmission and tell us who else in a family may be at risk.

Mutations lead to genetic conditions in the following ways. The first is through inherited disorders. Some inherited genetic diseases and conditions are single gene disorders (often described as classical inheritance disorders). They are caused by a mutation in a single gene or DNA sequence that is present in the germline and can therefore be passed from one generation to the next. The effect of the gene mutation dominates over other influences (such as environmental factors).

The inheritance patterns are described as autosomal if they are due to mutations in genes situated on one of the 22 autosomes, and sex linked if they occur on one of the sex chromosomes. For all these conditions, the mutation will be present from birth but disease onset may be later in life. Not all offspring will inherit the mutation, and examination of the family tree or pedigree may hint at the chance or likelihood of inheriting or being affected by a faulty gene. The same mutation (genotype) may be associated with different phenotypes—such as varying age at onset, symptoms, and disease severity or even no disease at all (processes known as anticipation, expressivity and penetrance describe these variations).

Autosomal dominant disorders are caused by a mutation on one of the genes in a pair. The single mutated gene dominates. As only one copy of the faulty gene is needed to produce the disorder, an affected parent has a 50% chance of passing the mutation on to a child. The mutation occurs on non-sex chromosomes, so males and females are usually affected in equal numbers. Examples are Huntington's Disease and a form of Haemochromatosis.

Autosomal recessive disorders are caused by a mutation on both genes in a pair. The condition only arises when each parent passes on a copy of the faulty gene. For most recessive conditions, people who only have one copy of the faulty gene are not affected but they are carriers. When both parents are carriers, each pregnancy carries a 25% chance that the child will be affected and a 50% chance that the child will be a carrier. Children sharing common ancestry are more likely to be affected due to higher rates of carriers in their background than those whose parents are from different backgrounds. These disorders usually affect males and females equally. Examples are Tay Sach's disease, cystic fibrosis, and sickle cell disease.

Sex linked disorders are caused by mutations on the sex chromosomes (X-linked and Y-linked disorders). The most common sex-linked conditions are X-linked recessive disorders carried in genes on the X chromosome, where females are carriers and half their male offspring are affected. This is because females have 2 copies of the X chromosome, so problems arising from a faulty X are usually “compensated” for by a normal X. Males only have one X chromosome inherited from their mother so if the X is faulty, the disease will be expressed. These conditions are never passed from an affected father to a son, but daughters can become carriers through inheritance of a faulty X from either an affected father or a carrier mother. Examples are Fragile X Syndrome and some forms of Muscular Dystrophy.

Other conditions arise from more complex patterns of inheritance (e.g. combinations of dominant and recessive genotypes, non-classical inheritance patterns such as mitochondrial disorders).

Chromosomal disorders are caused by errors in the number or structure of the chromosomes during the formation of sex cells but they are not necessarily passed through generations. These genetic abnormalities are common causes of mental retardation and physical defects in humans. Examples are Down's Syndrome (also known as Down Syndrome) which is most commonly caused by an extra copy (trisomy) of chromosome 21, and Turner's Syndrome, which involves only one copy (a monosomy) of the sex chromosomes—a single X.

Many common health conditions may be caused by a combination of inherited genetic mutations (polygenic) and other risk factors, and are known as multi-factorial or complex conditions. Examples include chronic adult diseases such as diabetes and rheumatoid arthritis and congenital conditions such as cleft palate.

The cause of these conditions is difficult to trace because there are many compounding factors. A combination of certain alleles, for example, or how a person responds to his/her environment, may lead to the condition in some cases but not in others. These conditions are often difficult to trace in a family because they have a low inheritance. For example, the chance that a sibling has the same polygenic condition as you is much lower than it would be if it was caused by a single gene.

Non-Mendelian or polygenic health effects may depend on two, three or many genetic loci, with greater or smaller contributions from environmental factors. We use multifactorial here as a catch-all term covering all these possibilities. More specifically, the genetic determination may involve a small number of loci (oligogenic) or many loci each of individually small effect (polygenic); or there may be a single major locus with a polygenic background.

Second, there are monogenic diseases or other health effects, the so-called Mendelian disorders. These diseases are those whose presence or absence depends on the genotype at a single locus. That is not to say that the character itself is programmed by only one pair of genes: expression of any human character is likely to require a large number of genes and environmental factors. However, sometimes a particular genotype at one locus is both necessary and sufficient for the character to be expressed, given the normal human genetic and environmental background.

Most genetic health effects are governed by genes at more than one locus. The further away a health effect is from the primary gene action, the less likely it is to show a simple Mendelian pedigree pattern. DNA sequence variants are virtually always cleanly Mendelian—which is their major attraction as genetic markers. Protein variants (electrophoretic mobility or enzyme activity) are usually Mendelian but can depend on more than one locus because of post-translational modification. The failure or malfunction of a developmental pathway that results in a birth defect is likely to involve a complex balance of factors. Thus the common birth defects (cleft palate, spina bifida, congenital heart disease, etc.) are rarely Mendelian. Behavioral traits like IQ test performance or schizophrenia are still less likely to be Mendelian—but they may still be genetically determined to a greater or lesser extent.

The genetic material used to produce the formulation may include any nucleotide sequence that has been associated with a health effect in humans. Examples of genetically influenced human health effects are provided in Table 1 below.

TABLE 1
Genetically Influenced Human Health Effects
Health Effect

Abdominal Aortic Aneurysm
Acid Reflux Disease
Acromegaly
Addiction
Addison's Disease
Aging
AIDS
Alcoholism
Allergies
Allograft Rejection
Alopecia Areata
Alpha-1 Antitrypsin Deficiency
Alzheimer's Disease
Amyotrophic Lateral Sclerosis
Angioedema
Ankylosing Spondylitis
Anorexia Nervosa
Anxiety Disorder
Arthritis
Asian Flush
Asthma
Atherosclerosis
Athletic Endurance
Atopic Dermatitis
Atrial Fibrillation
Attention Deficit Hyperactivity Disorder
Autism
Autoimmune Disease
Autoimmune Hepatitis
Baldness
Bardet-Biedl Syndrome
Basal Cell Carcinoma
Batten Disease
Behçet's Disease
Bicuspid Aortic Valve
Bipolar Disorder
Bladder Cancer
Blindness
Blood
Blood Pressure
Bloom Syndrome
Brain Aneurysm
Breast Cancer
Canavan Disease
Cancer
Cannabis Dependence
Carpal Tunnel Syndrome
Cataracts
Celiac Disease
Cerebrovascular Disease
Cervical Cancer
Charcot-Marie-Tooth
Cholesterol
Chondrodysplasia
Chorionic Plate Inflammation
Chronic Fatigue Syndrome
Chronic Kidney Disease
Chronic Lower Respiratory Disease
Chronic Obstructive Pulmonary Disease
Cleft Palate
Cluster Headaches
Colon Cancer
Colorectal Cancer
Conduct Disorder
Coronary Artery Disease
Crigler-Najjar Syndrome
Crohn's Disease
Cushing's Syndrome
Cystic Fibrosis
Deafness
Deep Vein Thrombosis
Dementia
Depression
Dermatomyositis
Diabetes (Type I and II)
Drug Abuse
Dyslexia
Dystonia
Earwax
Eczema
Emphysema
Endometrial Cancer
Endometriosis
Eosinophilic Esophagitis
Epilepsy
Fabry Disease
Factor XI Deficiency
Familial Dysautonomia
Familial Hypertrophic Cardiomyopathy
Familial Mediterranean Fever
Fanconi Anemia
Fast Twitch Muscle Response
Fibromyalgia
Gallbladder Cancer
Gallstone Disease
Gastric Cancer
Gastrointestinal Cancer
Gaucher's Disease
GERD
Gestational Diabetes
Gilbert Syndrome
Gingivitis
Glaucoma
Glioma
Glycogen Storage Disease Type 1A
Glycogen Storage Disease Type II
Gout
Graves' Disease
Group A Streptocococcal Infection
Hashimoto Thyroiditis
HDL Cholesterol
Hearing Loss
Heart Disease
Heartburn
Height
Hemochromatosis
Hemophilia
Hepatitis C
Herpes
High Blood Pressure
Hirschsprung Disease
HIV
Hodgkin Lymphoma
Homocystinuria
Hypercholesterolemia
Hypertriglyceridemia
In Vitro Fertilization
Infectious Diseases
Inflammatory Bowel Disease
Intracranial Aneurysm
Intrahepatic Cholestasis of Pregnancy
Ischemia
Kawasaki Disease
Kidney Stones
Lactose Intolerance
LDL Cholesterol
Leber Congenital Amaurosis
Leprosy
Leukemia
Li-Fraumeni Syndrome
Liver Cancer
Longevity
Lumbar Disc Disease
Lung Cancer
Lupus
Lyme Disease
Lymphoma
Lynch Syndrome
Lysosomal Storage Disease
Macular Degeneration
Malaria
Malignant Melanoma
Maple Syrup Urine Disease
Mednik Syndrome
Melanoma
Memory
Menarche, age at
Meniere's Disease
Meningioma
Mesothelioma
Migraines
Mucolipidosis Type IV
Multiple Sclerosis
Muscle Building
Muscular Dystrophy
Myasthenia Gravis
Myocardial Infarction
Myopia
Narcolepsy
Neuroblastoma
Neurodegenerative Disorder
Nicotine Dependence
Niemann-Pick Disease
Nonalcoholic Fatty Liver Disease
Non-Hodgkin Lymphoma
Obesity
Obsessive Compulsive Disorder
Oculopharyngeal Muscular Dystrophy
Osteoarthritis
Osteoporosis
Otitis
Otosclerosis
Ovarian Cancer
Pachyonychia Congenita Type I
Pancreatic Cancer
Panic Disorder
Parkinson's Disease
Pelvic Organ Prolapse
Peptic Ulcer Disease
Performance IQ
Periodontitis
Peripheral Arterial Disease
Phenylketonuria
Photic Sneeze Reflex
Polycystic Kidney Disease
Polycystic Ovary Syndrome
Post-Traumatic Stress Disorder
Pre-Eclampsia
Premature Birth
Primary Biliary Cirrhosis
Primary Sclerosing Cholangitis
Progressive Supranuclear Palsy
Prostate Cancer
Psoriasis
Psoriatic Arthritis
Resting Heart Rate
Restless Legs Syndrome
Rheumatic Fever
Rheumatoid Arthritis
Rhinitis
Rhinosinusitis
Sarcoidosis
Schizophrenia
Sciatica
Seasonal Affective Disorder
Secondhand Smoke Susceptibility
Sickle Cell Anemia
Sickle Cell Trait
Sjögren's Syndrome
Skin Cancer
Smell
Smoking
Social Anxiety Disorder
Speech
Spinal Muscular Atrophy
Split Hand
Squamous Cell Carcinoma
Stickler Syndrome
Stomach Cancer
Stroke
Stuttering
Sudden Infant Death Syndrome
Suicide
Sun Sensitivity
Systemic Sclerosis
Taste
Tay-Sachs Disease
Testicular Cancer
Thyroid Cancer
Torsion Dystonia
Tourette Syndrome
Tuberculosis
Tyrosinemia Type I
Ulcer
Ulcerative Colitis
Venous Thromboembolism
Vitamin D Insufficiency
Vitiligo
Von Gierke Disease
Von Willebrand Disease
Wilms Tumor

The specific genetic material that produces these health effects can be found in one of the following databases (this is a non-exhaustive list of databases that may be consulted). For example, the alleles or combination of alleles that influence human health may be stored in these databases. The entire contents of all of the following databases are incorporated herein by reference. The databases include:

dbGaP—genotype and phenotype database. This database includes phenotype and genotype data, as well as the associations between them. The information is obtained from genome-wide association studies, medical sequencing, and molecular diagnostic assays. It provides access to summaries of the genotype and phenotype data including coded individual-level phenotypes, genotypes, and pedigrees. The database is available at http://www.ncbi.nih.gov/gap.

dbSNP—single nucleotide polymorphism (SNP) database. The database is available at http://www.ncbi.nlm.nih.gov/nucest.

dbVar—genomic structural variation database. This database includes information associated with large scale genomic variation, including large insertions, deletions, translocations, and inversions. In addition to variation discovery, dbVar also includes associations of defined variants with phenotype information. The database is available at http://www.ncbi.nlm.nih.gov/dbvar.

DNA Data Bank of Japan (DDBJ)—Japanese nucleotide sequence database. The DDBJ is the nucleotide sequence database for Asia. The database is available at http://www.ddbj.nig.ac.jp/intro-e.html.

EMBL-Bank—nucleotide sequence database. This database is Europe's primary nucleotide sequence resource. It includes DNA and RNA sequences from direct submissions, genome sequencing projects, and patent applications. The database is available at http://www.ncbi.ac.uk/embl/.

Epigenomics—epigenetic maps and data sets. This database includes epigenomic datasets. It allows users to explore and visualize the datasets. The database is available at http://www.ncbi.nlm.nih.gov/epigenomics.

EST—expressed sequence tag (EST) records. This database includes all records found within the EST division of GenBank. EST records include first-pass single-read cDNA sequences and do not include annotated biological features. The database is available at http://www.ncbi.nlm.nih.gov/nucest.

European Nucleotide Archive (ENA)—collection of nucleotide sequence databases. The ENA provides a comprehensive record of the world's nucleotide sequencing information including raw sequencing data, sequence assembly information and functional annotation. The ENA is available at http://www.ebi.ac.uk/ena/.

GenBank—National Institute of Health's genetic sequence database. It is an annotated collection of all publicaly available DNA sequences. The database is available at http://www.ncbi.nlm.nih.gov/genbank/.

Genome—whole genome sequences. This database includes the entire genomes of a large number of organisms. The genomes represent both completely sequenced organisms and those for which sequencing is in progress. All three main domains of life—bacterial, archaea, and eukaryote—are represented. The database is available at http://www.nvbi.nlm.nih.gov/sites/entrez!db=genome.

Gene—gene centered information. This database contains information about the characteristics and defining sequences of genes from species in the Genome and RefSeq databases as well as other model organisms. The database is available at http://www.ncbi.nlm.nih.gov/gene.

GEO Profiles—expression and molecular abundance profiles. This database includes individual gene expression and molecular abundance profiles assembled from the Gene Expression Omnibus (GEO) repository. The database is available at http://www.ncbi.nlm.nih.gov/geoprofiles.

GSS—Genome Survey Sequence (GSS) records. This database includes all records found within the GSS division of GenBank. GSS records contain first-pass single-read genomic sequences and rarely include annotated biological features. The database is available at http://www.ncbi.nlm.nih.gov/nucgss.

GWAS Central—genetic association information. This database provides a centralized compilation of summary level findings from genetic association studies. It is build on a basal layer of markers that comprise all known SNPs and other variants from public databases. Allele and genotype frequency data, plus genetic association significance findings, are added on top of the marker data and organized the same way that investigations are reported in typical journal manuscripts. The database is available at http://www.gwascentral.org/index.

HapMap—catalog of common genetic variants that occur in humans. This database includes information about genetic variants such as what they are, where they are located in our DNA, and how they are distributed among people within populations and among populations in different parts of the world. HapMap is available at http://hapmap.ncbi.nlm.nih.gov.index.html.en.

Human Genome Mutation Database (HGMD)—The database is available at www.hgmd.org;

JSNP—Japanese SNP database. The database is available at http://snp.ims.u-tokyo.ac.jp/.

Nucleotide—core subset of nucleotide sequence records. The database contains records for all Entrez Nucleotide sequences that are not found within the EST or Genome Survey Sequence (GSS) divisions of GenBank. These include sequences from all remaining divisions of GenBank, NCBI Reference Sequences (RefSeq), Whole Genome Shotgun (WGS) sequences, Third Party Annotation (TPA) sequences, and sequences imported from the Entrex Structure database. The database is available at http://www.ncbi.nlm.nih.gov/nuccore.

Online Mendelian Inheritance of Man (OMIM)—This is a catalog of human genes and genetic disorders, with links to literature references, sequence records, maps, and related databases. It contains full-text, referenced overviews on all known mendelian disorders and over 12,000 genes. It focuses o the relationship between phenotype and genotype. This database is available at http://www.omim.org/ and http://www.ncbi.nlm.nih.gov/omim.

PopSet—population study data sets. This database includes DNA sequences that have been collected to analyze the evolutionary relatedness of a population. The database is available at http://www.ncbi.nlm.nih.gov/popset.

Reference Sequence (RefSeq) database—This database includes a comprehensive, integrated, non-redundant, well-annotated set of sequences, including DNA, transcripts, and proteins. The database is available at http://www.ncbi.nlm.nih.gov/RefSeq/.

UniGene—gene oriented clusters of transcript sequences. This database includes automatic partitions of GenBank sequences into a non-redundant set of gene-oriented clusters. Each UniGene cluster includes sequences that represent a unique gene, as well as related information such as the tissue types in which the gene has been expressed and map location. The database is available at http://www.ncbi.nlm.nih.gov/unigene.

UniSTS—markers and mapping data. This database includes information about markers, or sequence tagged sites (STS). It integrates marker and mapping data from public resources including GenBank, RHdb, GDB, and various human maps (Genethon genetic map, Marshfield genetic map, Whitehead RH map, Whitehead YAC map, Stanford RH map, NHGRI chr 7 physical map, WashU chrX physical map). The database is available at http://www.ncbi.nim.nih.gov/unists.

As noted above, one of the basic tenets of homeopathic medicine or other diluted treatment therapy is that the body's natural healing processes can be evoked or enhanced using a high dilution treatment that resembles the cause of the disease, illness, or condition. In this way, the diluted treatment can be used to treat, cure, or otherwise affect the condition. Consequently, these diseases and other health effects are produced by preparing a formulation that includes genetic material associated with such diseases, illnesses, or conditions. Other positive health effects can be that are genetically influenced, such as fast twitch muscle, speed, strength, etc., may be encouraged or enhanced using a formulation made from the genetic material that influences these effects.

A therapeutic formulation is prepared by first indentifying genetic material that can naturally support and/or nurture the body's ability to overcome or relieve symptoms of undesirable health conditions and/or promote the body's ability to enhance desirable health effects. Such genetic materials may be said to be associated with the condition or health effect to be treated. This can be done by consulting, among other things, the databases listed above. Typically, this involves identifying alleles that are associated with one or more conditions that the formulation will be used to treat.

A mixture is prepared that includes the desired genetic material. In one embodiment, the mixture includes alleles associated with the health effects in question. The mixture may be prepared by identifying DNA that contains one or more allele associated with a health effect, amplifying and purifying the alleles to produce a sufficient quantity of genetic material, and then combining the alleles together to produce the mixture. The alleles may be combined together in equal or unequal amounts.

Any suitable method may be used to accomplish any of these steps. For example, genetic amplification may be performed by traditional carrier organism replication, polymerase chain reaction (PCR), molecular cloning (via restriction enzymes or recombination), reverse transcriptase PCR, and the like.

According to one exemplary embodiment of the present exemplary system and method, the desired genetic material is created by creating a blanking sequence that is the same as the desired allele, such as a structure having 200 bases. The genetic material is then implanted into a carrier organism (e.g., E. coli) to replicate the genetic material. According to one exemplary embodiment, the genetic material identified for treatment is encoded into the genome of the carrier organism, which may be a virus or bacterium or any other organism in order to grow a sufficient amount of the genetic material.

According to another exemplary embodiment, the desired mass of genetic material is generated via polymerase chain reaction (PCR). The polymerase chain reaction (PCR) is a technique in molecular biology to amplify a single or few copies of a piece of DNA across several orders of magnitude, generating thousands to millions of copies of a particular DNA sequence. The method relies on thermal cycling, consisting of cycles of repeated heating and cooling of the reaction for DNA melting and enzymatic replication of the DNA. Primers (short DNA fragments) containing sequences complementary to the target region along with a DNA polymerase are key components to enable selective and repeated amplification. As PCR progresses, the DNA generated is itself used as a template for replication, setting in motion a chain reaction in which the DNA template is exponentially amplified.

According to yet another exemplary embodiment of the present exemplary system and method, the desired mass of genetic material is generated via molecular cloning. Molecular cloning refers to the procedure of isolating a defined DNA sequence and obtaining multiple copies of it in vivo. Cloning is frequently employed to amplify DNA fragments containing genes, but it can be used to amplify any DNA sequence such as promoters, non-coding sequences, chemically synthesized oligonucleotides, randomly fragmented DNA or any other DNA based genetic material.

The genetic material may include elements that will produce an epigenetic effect, including, but in no way limited to, DNA methylation. Epigenetics has been defined as “the study of the mechanisms of temporal and spatial control of gene activity during the development of complex organisms.” Thus epigenetic can be used to describe anything other than DNA sequence that influences the development of an organism. Two predominant epigenetic mechanisms are DNA methylation and histone modification.

The molecular basis of epigenetics involves modifications of the activation of certain genes, but not the basic structure of DNA. Additionally, the chromatin proteins associated with DNA may be activated or silenced. This accounts for why the differentiated cells in a multi-cellular organism express only the genes that are necessary for their own activity. Epigenetic changes are preserved when cells divide. Most epigenetic changes only occur within the course of one individual organism's lifetime, but, if a mutation in the DNA has been caused, some epigenetic changes are inherited from one generation to the next.

In the context of the present exemplary system and method, specific epigenetic processes include paramutation, bookmarking, imprinting, gene silencing, X chromosome inactivation, position effect, reprogramming, transvection, maternal effects, the progress of carcinogenesis, many effects of teratogens, regulation of histone modifications and heterochromatin, and technical limitations affecting parthenogenesis and cloning.

When sufficient genetic material is produced according to any methodology, including the methodologies mentioned above, the genetic material is then utilized as the initial compound, (also referred to herein as the initial substance or the initial ingredient) to prepare the formulation.

The highly diluted formulation may be made from the genetic material as follows. The process includes preparing a first mixture (also referred to herein as the initial mixture or mother tincture) that includes the genetic material, diluting the first mixture with a diluting agent, and potentizing or activating the first mixture by vigorously shaking it. The dilution and shaking steps are repeated multiple times until the desired potency is reached.

The first mixture is prepared by mixing the genetic material with a diluting agent. The diluting agent may be any suitable material such as ethanol, water, glycerin, or any combination of these materials. The diluting agent preferably includes ethanol since it typically forms a more stable solution that keeps for a longer time. Aqueous or other types of solutions may be preferable in situations where the genetic material is soluble in water but not ethanol or the genetic material is subject to chemical change or decomposition in ethanol.

In one embodiment, the diluting agent may include at least approximately 20% v/v ethanol, at least approximately 60% v/v ethanol, at least approximately 70% v/v ethanol, or at least approximately 90% v/v ethanol. The remainder of the diluting agent may be water, and preferably distilled water. In another embodiment, the diluting agent includes no more than approximately 10% v/v water, no more than approximately 30% v/v water, no more than approximately 40% v/v water, or no more than approximately 80% v/v water.

The first mixture may include any suitable amount of the genetic material. It should be appreciated that if the genetic material is prepared as part of a solution, any non-genetic material in the solution should be accounted for when determining the concentration of the genetic material in the first mixture. For example, if the genetic material is part of an aqueous solution that is combined with the diluting agent to form the first mixture, the water in the aqueous solution should be considered part of the non-genetic material in the first mixture.

In one embodiment, the first mixture includes no more than approximately ⅕ w/w or v/v genetic material or no more than approximately ⅛ w/w or v/v genetic material. In another embodiment, the first mixture includes at least 1/1000 w/w or v/v genetic material or at least 1/500 w/w or v/v genetic material. In yet another embodiment, the first mixture includes approximately ⅕ w/w or v/v genetic material to approximately 1/1000 w/w or v/v genetic material or approximately ⅛ w/w or v/v genetic material to approximately 1/500 w/w or v/v genetic material. Preferably, the first mixture includes approximately 1/10 w/w or v/v genetic material or 1/100 w/w or v/v genetic material.

In some situations, it may be desirable to subject the genetic material to a maceration process before combining it with the diluting agent to form the first mixture. The maceration process proceeds as follows. The genetic material is placed in a container such as a jar or bottle and a solvent is added until it completely engulfs the genetic material. The container is closed, placed in a dark room at room temperature and vigorously shaken at regular intervals. This is done for up to two months and then the liquid in the container is decanted.

In other situations, it may be desirable to subject the genetic material to a percolation process before combining it with the diluting agent to form the first mixture. The percolation process proceeds as follows. The genetic material is dried and reduced to a fine powder. A solvent is mixed with the powder until it is uniformly and distinctly damp. The damp powder is transferred to a percolator, allowed to stand for one hour, and then packed firmly into the percolator.

The percolator should be provided with a stop-cock or other device to control the flow through the unit. A plug of absorbent cotton is inserted into the neck above the stop-cock and covered with a filter material. The damp powder is spread onto the filter material and then the filter material and plug are pressed down with a broad, inert tamper. Another piece of filter material is placed on top of the existing filter material.

While holding the filter and plug combination down, pour the solvent upon the contents of the percolator until the filter and plug combination is covered, allowing the fluid to run gently down the rod so that the filter material is not displaced. Close the percolator to prevent evaporation. Close the valve or stop-cock as soon as the fluid begins to drop and allow it to stand 24 hours or longer depending on the nature of the contents. The fluid should pass through the percolator into the receiver, drop by drop, at a rate of approximately 10 to 30 drops per minute. Additional solvent should be periodically added to keep the liquid surface above the powder, thereby preventing access of air.

The genetic material may also be heated as part of the maceration or percolation processes or as part of another different process. The heat may cause the constituents of the genetic material to break down and lead to a more complex extraction of medicinal properties. The genetic material may be heated using any of a number of suitable techniques.

In one embodiment, the genetic material is incubated using the following process. The process is the same as that described for maceration above except that after the container is closed, it is heated up to 100° C. or up to 50° C. (e.g., approximately 37° C.) and maintained at the desired temperature, with occasional agitation, for approximately one hour. After cooling, the container is placed in a dark room and the maceration process proceeds as described above.

In another embodiment, the genetic material is heated using an infusion process. The dried genetic material and a solvent are placed in a container and allowed to stand for up to an hour (e.g., approximately 15 minutes). Boiling water is poured over the preparation and, under a reflux condenser, the contents are maintained at the boiling point for up to 30 minutes (e.g., approximately 5 minutes). The container is cooled to room temperature, closed, placed in a dark room at normal temperature, and vigorously shaken at regular intervals. The remainder of the process is similar to that described for maceration above.

In another embodiment, the genetic material is heated using a decoction process. The dried genetic material and a solvent are placed in a container and allowed to stand overnight. The contents are then heated under a reflux condenser and the boiling point is maintained for 30 minutes. After cooling, the container should be handled in the manner described for the infusion process.

The solvent mentioned above in connection with the maceration and percolation processes as well as the heating methods may be any suitable solvent. In one embodiment, the solvent may be the same as any of the diluting agents described above. Also, it should be appreciated that any of the heating methods described above can be used separately from the maceration or percolation processes. They can be used in conjunction with any other process or as a standalone heating step.

There may be some situations where the genetic material is not be soluble in the diluting agent. In these situations, the genetic material may be converted into a form that can be introduced into the core process. This is done by diluting the genetic material while it is in a solid or semi-solid form using a process called trituration. The genetic material is combined with a solid diluting agent to form a first solid mixture. The solid diluting agent can be any suitable material such as lactose or sucrose. Preferably, the solid diluting agent is largely or entirely lactose.

The genetic material and the solid diluting agent are mixed together in any suitable way using any suitable equipment. In one embodiment, the materials are processed by hand using a mortar and pestle. This method may be especially useful when processing smaller quantities. In another embodiment, the materials are processed using mechanical equipment such as a ball mill. This device includes a cylindrical porcelain jar fitted with a tight lid. The materials are placed in the jar together with very hard porcelain cylinders. The jar is closed, placed on horizontal rollers, and rotated by electric motors for a long enough time to ensure complete and thorough mixing of the materials (e.g., approximately two hours). This method may be especially useful when processing larger quantities.

The first solid mixture may include any suitable amount of the genetic material. In one embodiment, the first solid mixture includes no more than approximately ⅕ w/w genetic material or no more than approximately ⅛ w/w genetic material. In another embodiment, the first solid mixture includes at least 1/1000 w/w genetic material or at least 1/500 w/w genetic material. In yet another embodiment, the first solid mixture includes approximately ⅕ w/w genetic material to approximately 1/1000 w/w genetic material or approximately ⅛ w/w genetic material to approximately 1/500 w/w genetic material. Preferably, the first mixture includes approximately 1/10 w/w genetic material or 1/100 w/w genetic material.

The first solid mixture may be diluted further using the same process. The dilution ratio used for each successive, increasingly dilute solid mixture is preferably the same as the dilution ratio used to prepare the first solid mixture. However, the dilution ratio does not need to be the same and can potentially vary for each successive, increasingly dilute solid mixture.

Examples of suitable dilution ratios include 1:5 (it should be noted that this ratio is used by other dilution treatment methodologies but is not a proper dilution ratio in homeopathy), 1:10, 1:100, 1:1,000, 1:50,000, 1:100,000, 1:500,000, 1:1,000,000 or any ratio in between these. Since the materials are solid, the dilution ratio is preferably determined on a w/w basis. However, a w/v or v/v basis may be used as well to the extent practicable. The amount of genetic material in the increasingly dilute solid mixtures can be determined based on the original amount of genetic material in the first solid mixture, the number of times it was subsequently diluted, and the dilution ratio used each time.

One example of a trituration process is as follows. The first solid mixture is prepared by mixing the genetic material and the solid diluting agent at a ratio of 1:10 to form a mixture having a potency of 1×. It is mixed using a ball mill for two hours. The first solid mixture is diluted by mixing it with the solid diluting agent at a ratio of 1:10 and processing it in a ball mill to produce a second solid mixture having a potency of 2×. The second solid mixture is then diluted further by mixing it with the solid diluting agent at a ratio of 1:10 and processing it in a ball mill to produce a third solid mixture having a potency of 3×. The subsequent, increasingly dilute mixtures are all prepared using a dilution ratio of 1:10 to produce increasingly potent mixtures.

After one or more triturations, the solid mixture can be incorporated into the core process and used to prepare the first mixture referenced above. For example, the 3× trituration prepared above can be substituted for the raw genetic material used to prepare the first mixture. The 3× trituration readily dissolves in the diluting agent (e.g., ethanol, water, glycerin, etc.) and further dilution can proceed in the manner described below.

The first mixture is serially diluted to produce successive, increasingly dilute mixtures. The first mixture can be serially diluted any number of times using any suitable dilution ratio. The dilution ratio used to prepare each increasingly dilute mixture is preferably the same as the dilution ratio used to prepare the first mixture. However, the dilution ratio does not need to be the same and can potentially vary for each increasingly dilute mixture.

Examples of suitable dilution ratios include 1:5 (see previous note about this dilution ratio and its applicability to homeopathy), 1:10, 1:100, 1:1,000, 1:50,000, 1:100,000, 1:500,000, 1:1,000,000 or any ratio in between these. The dilution ratios may be on a w/w, w/v, or v/v basis. In one embodiment, the first mixture is diluted according to the decimal (X), centesimal (C), or fifty millesimal (LM) scale.

The final formulation may be labeled with a number followed by a roman numeral to indicate the final dilution and the manner in which the first mixture is serially diluted. Examples of such a label include 20X, 40C, and 20LM. The letter designation denotes the dilution ratio used in the process and the number before the letter indicates how many times the starting material has been diluted at that ratio. For example, V, X, C, and LM mean that each increasingly dilute mixture is prepared using a 1:5, 1:10, 1:100, and 1:50000 dilution ratio, respectively. The concentration can be determined by the number of dilutions given at the specified dilution ratio.

For example, a formulation labeled 40X has a concentration of 1×10⁻⁴⁰ and a formulation labeled 20C has the same concentration 1×100⁻²⁰ or 1×10⁻⁴⁰. Although the final concentration is the same, the formulations are not the same because the 40X formulation is prepared by undergoing 40 separate dilutions at a 1:10 dilution ratio and the 20C formulation is prepared by undergoing 20 separate dilutions at a 1:100 dilution ratio.

The designation M is also used as a potency designation on labels. However, the M is not a separate dilution ratio (like X, C and LM). It is merely shorthand for 1000C. The further dilution of a 1M potency includes serial 1:100 dilutions until the 2000^{th} potency is reach, which is designated 2M. Thus, 10M means 10000C, 15M means 15000C, and so forth.

In one embodiment, the first mixture is serially diluted using an average dilution ratio of no more than approximately 1:5 or 1:10. It should be appreciated that the phrase “no more than” is used in the context of the decimal value of the dilution ratio and not the Roman numeral notation used to refer to the dilution scale. For example, the decimal value of 1:5 is 0.2. The decimal value of the average dilution ratio is no more than approximately 0.2 but may be less than approximately 0.2 such as 1:10 (0.1) or 1:100 (0.01). Although the roman numeral notation increases as the decimal value of the dilution ratio drops, the decimal value is being referenced unless noted otherwise.

In another embodiment, the first mixture is serially diluted using an average dilution ratio of approximately 1:5 to approximately 1:1000000, approximately 1:10 to approximately 1:50000, or approximately 1:10 to approximately 1:100. The average dilution ratio refers to the average of all the dilution ratios used to serially dilute the first mixture. In situations where the same dilution ratio is used for each serial dilution, the average dilution ratio is the same as the dilution ratio used.

In another embodiment, the dilution ratio for each serial dilution of the first mixture is no more than approximately 1:5 or 1:10. In yet another embodiment, the dilution ratio for each serial dilution of the first mixture is approximately 1:5 to approximately 1:1000000, approximately 1:10 to approximately 1:50000, or approximately 1:10 to approximately 1:1000.

The diluting agent used to prepare each increasingly dilute mixture may the same as or different than the diluting agent used to prepare the first mixture. In one embodiment, the final formulation may be in the form of a solid tablet, pellet, or the like. The diluting agent includes at least approximately 50% w/w or v/v ethanol or at least approximately 70% w/w or v/v ethanol. In another embodiment, the final formulation is a liquid that is administered orally. The diluting agent used to dilute the first mixture and prepare a second mixture includes at least approximately 50% w/w or v/v ethanol or at least approximately 60% w/w or v/v ethanol. The diluting agent used to prepare the remainder of the increasingly dilute mixtures includes at least approximately 10% w/w or v/v ethanol or at least approximately 20% w/w or v/v ethanol.

The first mixture may be diluted using any suitable method. Two methods that may be used are the Hahnemannian and Korsakovian methods. The difference between the methods centers on whether the container is changed each time the mixture is diluted. The container is changed each time in the Hahnemannian method but is not in the Korsakovian method.

For example, a 3C formulation is made using the Hahnemannian method as follows. The 1C formulation is prepared by removing 1 part of the first mixture from its container and adding it to 99 parts of diluting agent in another container. The 2C formulation is prepared by removing 1 part of the 1C formulation from its container and adding it to 99 parts of diluting agent in yet another container. The 3C formulation is prepared by removing 1 part of the 2C formulation from its container and addition it to 99 parts of diluting agent in yet another container.

In contrast, a 3C formulation is made using the Korsakovian method in the same container. The 1C formulation is prepared by emptying the contents of the first mixture from the container so that 1 part remains (e.g., the small amount left on the walls and bottom of the container when it is emptied) and adding 99 parts diluting agent to the container. The 2C formulation is prepared by emptying the contents of the 1C formulation from the container so that 1 part remains and adding 99 parts diluting agent. This process is repeated again to produce the 3C formulation. In Korsakovian method every subsequent dilution is achieved by emptying the container of 99% on a w/w or v/v basis of the previous formulation and refilling it with fresh diluting agent.

An H or a K can be added to the label to indicate which method was used to produce the formulation. For example, 3CH indicates centesimal attenuation, Hahnemannian style. 3CK indicates centesimal attenuation, Korsakovian style.

A formulation can be prepared using the same method throughout or by combining the two methods or any other suitable method. In one embodiment, the Hahnemannian method is used for the first 12 to 200 serial dilutions and the Korsakovian method is used for additional dilutions. In another embodiment, the Hahnemannian method is used to prepare formulations up to 200C and the Korsakovian method is used to prepare formulations above 200C. For formulations above 200C, the Korsakovian method may be used for all of the serial dilutions or the Hahnemannian method may be used for each serial dilution up to 200C and then the Korsakovian method used thereafter.

Each successive, increasingly dilute mixture is potentized or activated by vigorously shaking the container holding the mixture. This vigorous shaking is known as succussion. Substances that are diluted without being vigorously shaken do not share the same healing property as succussed substances. Vigorously shaking the solution allows the formulation to remain potent past the point where none of the original molecules of genetic material remain in the dilution. The purely chemical effect of the genetic material is lost as it is diluted more and more, but with vigorous shaking the homeopathic effects are released. With vigorous shaking, the homeopathic remedy gets stronger and longer lasting with each successive dilution.

In one embodiment, each increasingly dilute mixture is succussed by subjecting it to vigorous shaking and an impact force. If the mixture is succussed by hand, this can be done by striking the container against an object such as a large book. If the mixture is succussed in an automated fashion, this can be done by a special mechanical shaking device. The device shakes the container and subjects it to an impact force.

In one embodiment, each increasingly dilute mixture is subjected to at least approximately 2 impact forces, at least approximately 5 impact forces, or at least approximately 10 impact forces. In another embodiment, each increasingly dilute mixture is subjected to approximately 2 to approximately 1000 impact forces, approximately 5 to approximately 100 impact forces, approximately 10 to approximately 50 impact forces, or approximately 20 to approximately 40 impact forces. The increasingly dilute mixtures may each be subjected to same number of impact forces or a different number of impact forces.

Each increasingly dilute mixture may be vigorously shaken for any amount of time that is desirable. In one embodiment, each increasingly dilute mixture is shaken for at least approximately 2 seconds, at least approximately 4 seconds, or at least approximately 8 seconds. In another embodiment, each increasingly dilute mixture is vigorously shaken for no more than approximately 2 hours, no more than approximately 1 hour, or no more than approximately 30 minutes. In yet another embodiment, each increasingly dilute mixture is vigorously shaken for approximately 2 seconds to approximately 2 hours, approximately 4 seconds to approximately 1 hour, or approximately 8 seconds to approximately 30 minutes. The increasingly dilute mixtures may each be vigorously shaken for the same amount of time or a different amount of time.

Each increasingly dilute mixture may be succussed by repeatedly starting and stopping the shaking. In one embodiment, each mixture is vigorously shaken at least approximately 2 times, at least approximately 5 times, or at least approximately 8 times. In another embodiment, each mixture is vigorously shaken no more than approximately 1000 times, no more than approximately 500 times, or no more than approximately 100 times. In yet another embodiment, each mixture is vigorously shaken approximately 2 to approximately 1000 times, approximately 5 to approximately 500 times, or approximately 8 to approximately 100 times.

It may be desirable to pause between shaking successive mixtures. In one embodiment, there is at least 1 minute, at least 2 minutes, or at least 3 minutes between shaking of each successive mixture. It should be appreciated that the pause between shaking successive mixtures may be any suitable length of time.

Formulations prepared using higher dilution ratios may require multiple dilutions between shaking For example, a formulation is prepared using the 1:50000 dilution ratio as follows. The genetic material is added as a liquid or a solid to lactose in a proportion of 1:100. If liquid, the genetic material is added as using a dropper or other dispenser to the lactose. The mixture is then triturated to the 3C trituration in the manner described above. A portion of the trituration, e.g., 0.062 g, is added to 500 drops of diluting agent in a container. One drop of the resulting mixture is then added to 2 ml of diluting agent. The mixture is then shaken for the first time to form the 1LM formulation.

The 2LM formulation is prepared by mixing one drop of the 1LM mixture with 0.575 g #10 pellets (500 #10 pellets) to form medicated pellets. One of the medicated pellets is added to 2 ml of diluting agent. The mixture is shaken to form the 2LM formulation. This process is repeated until the desired dilution level has been achieved.

The final formulation may have any suitable concentration of the first mixture or the genetic material. In one embodiment, the concentration of either the first mixture or the genetic material in the final formulation is no more than approximately 1×10⁻³ on a w/w or v/v basis, no more than approximately 1×10⁻⁴ on a w/w or v/v basis, no more than approximately 1×10⁻⁵ on a w/w or v/v basis, or no more than approximately 1×10⁻⁶ on a w/w or v/v basis.

The potency of the final formulation is different than its concentration. In homeopathy, the potency increases as it becomes increasingly dilute. A formulation having a higher concentration of genetic material has a lower potency than one that's more diluted. The potency of the formulation is given by the label. For example, a 15X formulation is more dilute and, therefore, has a higher potency than a 10X formulation. Likewise, a 10C formulation has a higher potency than a 15X formulation (10C=20X). The potency of the final formulation is at least 1V, 1X, or, desirably, 2X.

The final formulation may have any potency referenced herein. In one embodiment, different potency chords may be prepared from the first mixture. For example, the first mixture can be used to create potencies of 3X, 6X, 12X, 100X, 200X, etc. These are referred to as potency or dilution chords because the different potencies are made from the same starting mixture. Any desirable potency chords may be prepared using any suitable dilution ratio or scale. In one embodiment, potency chords may be prepared

The final formulation may be orally ingested by the patient in the form of a liquid, pellet or globule, or tablet. The liquid form may be packaged in any suitable container such as an amber glass bottle. It may also be dispensed from the container in any suitable manner such as with a dropper. The container may be any suitable size but preferably includes approximately 10 ml to approximately 100 ml of the final formulation or approximately 15 ml to approximately 60 ml of the final formulation. In another embodiment, the container includes approximately 10 ml, approximately 15 ml, approximately 30 ml, or approximately 60 ml of the final formulation.

The liquid form typically includes a mixture of purified water and ethanol, although it can include any combination of diluting agent and/or genetic material. The ethanol may be included to preserve the formulation and protect it from decomposition. In one embodiment, the final formulation includes no more than approximately 90% w/w or v/v ethanol, no more than approximately 75% w/w or v/v ethanol, no more than approximately 50% w/w of v/v ethanol, or no more than approximately 30% w/w or v/v ethanol. In another embodiment, the final formulation includes approximately 20% w/w or v/v ethanol, approximately 10% w/w or v/v ethanol, or approximately 5% w/w or v/v to approximately 25% w/w or v/v ethanol.

The pellet form is popular because it is easy to store and dispense. The diluting agent that makes up most of the pellet is sucrose, lactose, and/or other suitable polysaccharides. The pellets may be any suitable size and shape. In one embodiment, the pellets have a spherical shape and the size is designated according to the diameter of 10 pellets measured in millimeters. Standard sizes include very small pellets (#10), small pellets (#20), regular pellets (#35), and large pellets (#55). Pellets made of lactose will absorb alcoholic dilutions having a much larger percentage of water than will those made of sucrose.

The pellets may be medicated in any suitable way. In one embodiment, the pellets are medicated by placing them in a container and adding the last liquid formulation in a proportion of not less than 1% v/w (i.e., 1 drop of liquid for 2 g of unmedicated pellet). The pellets are allowed to soak for 3-5 minutes and then shaken to obtain the final formulation. The medicated pellets are dried at a temperature that is no more than approximately 40° C. This method may be especially suitable for situations where the liquid includes ethanol. If sucrose pellets are medicated then the liquid mixture should includes at least 70% w/w or v/v ethanol to prevent it from dissolving.

The pellets may be ingested sublingually (under the tongue) and allowed to dissolve for optimal absorption and utilization. They should be taken when there aren't other substances in the patient's mouth such as food, residues of tooth paste, mouth wash, gum, or the like. If the patient has recently eaten or had something in his or her mouth, then it may be desirable to have the patient wait approximately 1 hour before taking any pellets.

The tablet form may also be used to deliver the final formulation to the patient. Tablets differ from pellets based on how they are made and, in many situations, what they look like. The tablets can be made using any suitable process, although they are usually made using a different process than that used to make the pellets. Also, although the tablets and pellets may have any suitable shape, the pellets typically have a spherical shape and tablets have a non-spherical shape. Two examples of suitable tables include tablet triturates and compressed tablets.

Tablet triturates are soft, molded tablets produced from moist material on a triturate mold which gives them the shape of cut sections of a cylinder. They dissolve immediately when put in the patient's mouth. Tablet triturates are typically made using the following four step process. However, it should be appreciated that this process can be modified in a number of different ways and still produce a tablet that qualifies as a tablet triturate.

The first step in the method is to prepare a triturate having the desired potency in the manner described above. The second step is to add binder material to the mixture in any suitable amount (e.g., approximately 0.5 to approximately 2 parts binder to approximately 10 to approximately 20 parts triturate). The binder material may include any suitable material. In one embodiment, the binder material provided as a solution that includes a binder (e.g., such as gum arabic or microcrystalline cellulose), an optional preservative, an inert lubricant, and purified water. The third step is to mold the tablets by hand or with suitable equipment. The fourth step is to dry the molded tablets at a temperature of 70° F. to 110° F.

Compressed tablets are hard tablets that do not dissolve immediately when put in the patient's mouth. These are typically meant to be swallowed with water because they take too long to dissolve orally. Compressed tablets are formed by preparing a triturate having the desired potency in the manner described above. A binder material that is similar to or the same as that described in connection with the table triturates can be added to the triturate. The mixture is then compressed to form a hard tablet that is similar to conventional medicine tablets.

The final formulation can also be administered in the form of a capsule. The final formulation may be a liquid or a solid (e.g., a powder) that is enclosed in the capsule and orally administered to the patient. The capsule dissolves in the patient's stomach and releases the final formulation.

In addition to liquids, pellets, tablets, and capsules, the final formulation may also be provided in the form of ointments, lotions, and gels, which can be applied externally. These typically have less therapeutic effect than internally consumed remedies. The final formulation may also be provided as a suppository.

In one embodiment, the final formulation is part of a homeopathic remedy. The final formulation is provided in any of the forms discussed above and packaged in any suitable container. A label is attached to the container that communicates to user that the formulation inside is homeopathic in nature and/or includes genetic material of some potency such as at least 1X, at least 3X, and so forth.

It should be appreciated that the label does not need to use the words “genetic material” to communicate that genetic material is included in the formulation. Rather, the label can use a number of terms and descriptions to communicate this to the user. For example, the label may state that it includes one or more specific materials that qualify as genetic material or that the formulation is useful for supporting genetic health. There are numerous other ways the label can communicate this to the user.

The concepts described herein may also be applied to imprinting or digital homeopathy. The underlying concept is that vibrational exchange is the language of biochemistry. Molecules produced by the body that govern physiology and molecules administered as a therapeutic treatment work by transmitting an electromagnetic signal or signature, vibrating at a specific frequency, termed “resonance frequency,” that can be sensed and responded to by the cells in the body. Therapeutic treatments work by getting close enough to the cell so that their resonance frequencies can be picked up and responded to.

It follows, then, that it may not be necessary to administer a physical substance to the patient. The patient can be directly influenced through application of the resonance frequency. Instead of administering the actual substance, its resonance frequency is determined and applied to the patient in a concentrated, or potentiated, form.

The physics by which serial dilution concentrates frequencies is difficult to understand. Suffice it to say that removing from solution a molecule that once emitted a frequency creates an entity called a hyperproton, basically concentrated energy. The frequency given off from a given therapeutic agent can be recorded, digitized, emitted or imprinted into a liquid medium and then given to a biological system to generate a biological effect—the same effect that would occur if the original molecule was administered in intact form.

The resonance frequency of the genetic material (e.g., single SNPs or mixtures) can be identified and used for imprinting purposes. In one embodiment, the resonance frequencies for various genetic materials and/or final formulations may be stored in a computer database. The frequencies can be transmitted into the patient's body via any suitable transmission system for the purpose of evaluating which frequencies are the most valuable or beneficial to the patient's body. The specific frequencies that your body finds of value can then be imprinted into a carrier solution. The patient then places one or more drops of the imprinted solution under his/her tongue. The specific frequencies enter the patient's body, distribute through the patient's energetic nervous system, and stimulate the patient's cells to respond. Alternatively, the specific frequencies may be applied directly to the patient.

It should be appreciated that any source of electromagnetic energy can be used to identify the resonance frequency of the genetic materials and/or final formulations, imprint the carrier solution, or directly treat the patient. Examples of suitable sources of electromagnetic energy include RF, lasers, and the like.

In one embodiment, the final formulation may be used as part of a therapeutic treatment that includes measuring the electromagnetic signals of the genetic material in the formulation. For example, a homeopathic practitioner may measure a patient's energy field and then match that to the electromagnetic signature of a specific formulation to arrive at the appropriate treatment.

In another embodiment, the final formulation may be used as part of a therapeutic treatment such as that described in U.S. Pat. No. 6,142,927, titled “Method and Apparatus for Treatment with Resonant Signals,” issued on 7 Nov. 2000, which is hereby incorporated by reference in its entirety. Specifically, the final formulation may be used to provide digital sequences that are stored in the computer as disclosed in the '927 patent.

### EXAMPLES

The following examples are provided to further illustrate the disclosed subject matter. They should not be used to constrict or limit the scope of the claims in any way.

### Example 1

### Homeopathic Obesity Remedy

A homeopathic remedy for obesity is prepared as follows. The remedy is prepared using alleles that are believed to affect human susceptibility to obesity. The specific alleles used in the remedy are shown in Table 2, which is a non-exhaustive list of alleles that influence obesity in some manner. The alleles may represent an increased susceptibility to obesity or an increased resistance to obesity.

TABLE 2
Obesity Related Alleles

| | Risk/Benefit | |
|---|---|---|
| SNP | Allele | Gene |
| rs9939609 | A | FTO |
| rs3751812 | T | FTO |
| rs6235 | C | PCSK1 and PCSK2 |
| rs2568958 | A | NEGR1 |
| rs10913469 | C | SEC16B |
| rs7561317 | G | TMEM18 |
| rs7647305 | C | ETV5 |
| rs2844479 | T | AIF1-NCR3 |
| rs6265 | G | BDNF |
| rs7138803 | A | FAIM2 |
| rs4788102 | A | SH2B1 |
| rs12970134 | A | MCR4 |
| rs29941 | C | KCTD15 |

Genetic material is obtained directly from human samples that are obese. Individuals with the targeted ailment are screened for the SNPs shown in Table 2. Once an individual is found with the risk allele for a specific SNP through screening via PCR and DNA sequencing, the gene region holding the SNP is once again amplified via PCR multiple times over to obtain a high concentration of the gene section. The PCR product is cleaned using exonuclease and shrimp alkaline phosphate (EXOSAP) protocol to remove undesirable lengths of DNA from the tube, leaving purified copies of the gene segment or genomic locus in high concentrations to make the first mixture. The amount of genetic material in the PCR product is 200 nanograms/microliter.

The genetic material is mixed with a liquid diluting agent that includes 80% v/v ethanol and 20% v/v water at a ratio of 1:10 (i.e., 1 part genetic material to 9 parts diluting agent) to produce the first mixture. The first mixture is then serially diluted using the Hahnemannian method and the same diluting agent at a dilution ratio of 1:10 for each step. Each increasingly dilute mixture is vigorously shaken to potentize it. The final liquid mixture has a potency of 20XH.

Medicated pellets are prepared by placing 10 g of unmedicated sucrose pellets into a container and adding 5 drops of the final liquid mixture. The pellets are allowed to soak for 5 minutes and then shaken. The medicated pellets are dried at room temperature and packaged in an amber colored glass bottle. The dried medicated pellets constitute the final formulation.

### Example 2

### Homeopathic Type-2 Diabetes Remedy

A homeopathic remedy for Type-2 diabetes is prepared as follows. The remedy is prepared using alleles that are believed to affect human susceptibility or resistance to obesity. The specific alleles used in the remedy are shown in Table 3. The genetic material is obtained using the methods described in Example 1.

TABLE 3
Type-2 Diabetes Related Alleles

| | Risk/Benefit | |
|---|---|---|
| SNP | Allele | Gene |
| rs7903146 | T | TCF7L2 |
| rs12255372 | T | TCF7L2 |
| rs4506565 | T | TCF7L2 |
| rs9300039 | C | Intergenic |
| rs4402960 | T | IGF2BP2 |
| rs7754840 | C | CDKAL1 |
| rs7756992 | G | CDKAL1 |
| rs10811661 | T | CDKN2A/B |
| rs8050136 | A | FTO |
| rs2237895 | C | KCNQ1 |
| rs2283228 | | KCNQ1 |
| rs1801282 | C | PPARG |
| rs13266634 | C | SLC30A8 |
| rs10923931 | T | NOTCH2 |
| rs5219 | T | KCNJ11 |
| rs864745 | T | JAZF1 |
| rs1111875 | C | HHEX |
| rs4607103 | C | ADAMTS9 |
| rs4430796 | A | TCF2 |

The first mixture is prepared by combining the genetic material with a liquid diluting agent that includes 60% v/v ethanol and 40% v/v water at a ratio of 1:100 (i.e., 1 part genetic material to 99 parts diluting agent). The first mixture is then serially diluted using the Hahnemannian method and the same diluting agent with each increasingly dilute mixture being vigorously shaken until the final liquid mixture has a potency of 30C. The final liquid mixture is the final formulation, which is packaged in an amber colored bottle with a dropper.

### Example 3

### Homeopathic Breast Cancer Remedy

A homeopathic remedy for breast cancer is prepared as follows. The remedy is prepared using alleles that are believed to affect human susceptibility or resistance to breast cancer. The specific alleles used in the remedy are shown in Table 4. The genetic material is obtained using the methods described in Example 1.

TABLE 4
Breast Cancer Related Alleles

| | Risk/Benefit | |
|---|---|---|
| SNP | Allele | Gene |
| rs3803662 | T | TNRC9 |
| rs1045485 | G | CASP8 |
| rs13387042 | A | Intergenic |
| rs2981582 | A | FGFR2 |
| rs889312 | A | MAP3K1 |
| rs3817198 | T | LSP1 |
| rs13281615 | T | Intergenic |
| rs1799950 | G | BRCA1 |
| rs4986850 | A | BRCA1 |
| rs2227945 | G | BRCA1 |
| rs16942 | G | BRCA1 |
| rs1799966 | G | BRCA1 |
| rs766173 | G | BRCA2 |
| rs144848 | G | BRCA2 |
| rs4987117 | T | BRCA2 |
| rs1799954 | T | BRCA2 |
| rs11571746 | C | BRCA2 |
| rs11571747 | C | BRCA2 |
| rs4987047 | T | BRCA2 |
| rs11571833 | T | BRCA2 |
| rs1801426 | G | BRCA2 |
| rs3218707 | C | ATM |
| rs4987945 | G | ATM |
| rs4986761 | C | ATM |
| rs3218695 | A | ATM |
| rs1800056 | C | ATM |
| rs1800057 | G | ATM |
| rs3092856 | T | ATM |
| rs1800058 | T | ATM |
| rs1801673 | T | ATM |
| rs17879961 | C | CHEK2 |
| rs1042522 | C | TP53 |
| rs1799750 | 2G | MMP1 |
| rs3918242 | T | MMP9 |
| rs3218536 | A | XRCC2 |
| rs1219648 | G | FGFR2 |
| rs2981578 | G | FGFR2 |
| rs2981582 | T | FGFR2 |
| rs3135718 | G | FGFR2 |
| rs7895676 | C | FGFR2 |
| rs713041 | T | GPX4 |
| rs757229 | | GPX4 |
| rs438034 | T | CENPF |
| rs2056116 | G | Intergenic |
| rs3218005 | A | Intergenic |
| rs2854344 | A | RB1 |
| rs2268578 | T | LUM |
| rs4151620 | C | RB1 |
| rs351855 | T | FGFR4 |
| rs361525 | A | TNF |
| rs1801270 | A | CASP8 |
| rs203462 | | AKAP10 |
| rs1042638 | | TPD52 |
| rs997669 | A | CCNE1 |
| rs3176336 | A | CDKN1A |
| rs34330 | C | CDKN1B |
| rs3731239 | C | CDKN2A |
| rs4415084 | T | Intergenic |

The first mixture is prepared by combining the genetic material with a liquid diluting agent that includes 88% v/v ethanol and 12% v/v water at a ratio of 1:10 (i.e., 1 part genetic material to 9 parts diluting agent). The first mixture is then serially diluted using the Hahnemannian method and the same diluting agent with each increasingly dilute mixture being vigorously shaken until the final liquid mixture has a potency of 60X.

Medicated pellets are prepared as follows. 10 ml of the final liquid mixture is added to a thousand grams of sucrose pellets. The pellets are thoroughly shaken to distribute the liquid mixture evenly. The pellets are soaked for 5 minutes and then dried and packaged in containers that hold about eighty standard sized pellets.

### Illustrative Embodiments

Reference is made in the following to a number of illustrative embodiments of the disclosed subject matter. The following embodiments illustrate only a few selected embodiments that may include one or more of the various features, characteristics, and advantages of the disclosed subject matter. Accordingly, the following embodiments should not be considered as being comprehensive of all of the possible embodiments. The concepts and aspects of one embodiment may apply equally to one or more other embodiments or may be used in combination with any of the concepts and aspects from the other embodiments. Any combination of any of the disclosed subject matter is contemplated.

In one embodiment, a method comprises mixing human genetic material and a diluting agent to produce a first mixture and serially diluting at least a portion of the first mixture to produce a first formulation. Serially diluting at least a portion of the first mixture may include producing successive, increasingly dilute mixtures and vigorously mixing each successive mixture. Serially diluting at least a portion of the first mixture may include producing successive, increasingly dilute mixtures and succussing each successive mixture.

Serially diluting at least a portion of the first mixture may include producing successive, increasingly dilute mixtures and vortexing each increasingly dilute mixture. Serially diluting at least a portion of the first mixture may include producing successive, increasingly dilute mixtures using the same dilution ratio for each increasingly dilute mixture.

The human genetic material may be associated with a health effect in humans. The human genetic material may include an allele that is associated with a health effect in humans. The human genetic material may be non-native genetic material.

The first mixture may be serially diluted using an average dilution ratio of no more than approximately 1:5. The first mixture may be serially diluted using an average dilution ratio of approximately 1:5 to approximately 1:1000. The dilution ratio for each serial dilution of the first mixture may be no more than approximately 1:5. The dilution ratio for each serial dilution of the first mixture may be approximately 1:5 to approximately 1:1000.

The first mixture may be serially diluted at least 3 times. The first mixture may be serially diluted at least 5 times. The concentration of the human genetic material in the first formulation may be no more than 1×10⁻³ w/w or v/v. The concentration of the human genetic material in the first formulation may be no more than 1×10⁻⁶ w/w or v/v. The diluting agent may be water, alcohol, glycerin, lactose, and/or sucrose. The first mixture may be serially diluted with water, alcohol, glycerin, lactose, and/or sucrose.

According to another embodiment, a method comprises mixing genetic material and a diluting agent to form a first mixture, repeatedly diluting at least a portion of the first mixture to produce successive, increasingly dilute mixtures, and succussing each increasingly dilute mixture. Succussing each increasingly dilute mixture may include subjecting each increasingly dilute mixture to an impact force. The same dilution ratio may be used to produce each increasingly dilute mixture.

The genetic material may include human genetic material. The genetic material may be associated with a health effect in humans. The genetic material may include an allele that is associated with a health effect in humans. The genetic material may be non-native genetic material.

Each increasingly dilute mixture may be diluted using an average dilution ratio of no more than approximately 1:5. Each increasingly dilute mixture may be diluted using an average dilution ratio of approximately 1:5 to approximately 1:1000. The dilution ratio for each increasingly dilute mixture may be no more than approximately 1:5. The dilution ratio for each increasingly dilute mixture may be approximately 1:5 to approximately 1:1000.

Repeatedly diluting at least a portion of the first mixture may produce at least 3 increasingly dilute mixtures. Repeatedly diluting at least a portion of the first mixture may produce at least 5 increasingly dilute mixtures. The concentration of the genetic material in the last increasingly dilute mixture may be no more than 1×10⁻³ w/w or v/v. The concentration of the genetic material in the last increasingly dilute mixture may be no more than 1×10⁻⁶ w/w or v/v. The diluting agent may be water, alcohol. glycerin, lactose, and/or sucrose. Each increasingly dilute mixture may be diluted with water, alcohol, glycerin, lactose, and/or sucrose.

According to another embodiment, a formulation may be produced using any of the methods or combination of steps from the methods disclosed herein. The formulation may comprise instructions for oral ingestion of the formulation by a human. The formulation may be a homeopathic remedy.

According to another embodiment, a method comprises obtaining a genetic sample from a patient, identifying genetic material associated with a health effect in the patient, and orally administering a formulation that is prepared from the genetic material to the patient. The method may also comprise mixing the genetic material and a diluting agent to form a first mixture and repeatedly diluting at least a portion of the first mixture to produce successive, increasingly dilute mixtures. The formulation includes one of the increasingly dilute mixtures.

The genetic material may include an allele that is associated with a health effect in humans. The genetic material may be non-native genetic material. The concentration of the genetic material in the formulation may be no more than 1×10⁻³ w/w or v/v. The concentration of the genetic material in the formulation may be no more than 1×10⁻⁶ w/w or v/v.

According to another embodiment, a homeopathic remedy comprises a container, a formulation inside the container, and a label attached to the container. The label may communicate that the formulation includes non-native human genetic material at a potency of at least 1X. The label may communicate that the formulation includes non-native genetic material at a potency of at least 3X. The label may communicate that the formulation includes non-native genetic material at any potency from 1V to 1000000 LM and any in between. The label may communicate that the formulation includes a risk allele associated with a human health effect. The label may communicate that the formulation is homeopathic in nature. The formulation may be in the form of a liquid, pellet, table, or capsule (or any other suitable form).

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure the term shall mean,” etc.).

References to specific examples, use of “i.e.,” use of the word “invention,” etc., are not meant to invoke exception (b) or otherwise restrict the scope of the recited claim terms. Other than situations where exception (b) applies, nothing contained herein should be considered a disclaimer or disavowal of claim scope. The subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be given their broadest interpretation in view of the prior art and the meaning of the claim terms.

As used herein, spatial or directional terms, such as “left,” “right,” “front,” “back,” and the like, relate to the subject matter as it is shown in the drawings. However, it is to be understood that the described subject matter may assume various alternative orientations and, accordingly, such terms are not to be considered as limiting. Furthermore, articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y). Likewise, as used herein, the term “and/or” shall also be interpreted to be inclusive (e.g., “x and/or y” means one or both x or y). In situations where “and/or” or “or” are used as a conjunction for a group of three or more items, the group should be interpreted to include one item alone, all of the items together, or any combination or number of the items. Moreover, terms used in the specification and claims such as have, having, include, and including should be construed to be synonymous with the terms comprise and comprising.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques. Moreover, all ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

What is claimed is:

1. A method comprising:
mixing human genetic material and a diluting agent to produce a first mixture; and
serially diluting at least a portion of the first mixture to produce a first formulation;
wherein the human genetic material is non-native genetic material.

2. The method of claim 1 wherein serially diluting at least a portion of the first mixture includes producing successive, increasingly dilute mixtures and vigorously mixing each increasingly dilute mixture.

3. The method of claim 1 wherein serially diluting at least a portion of the first mixture includes producing successive, increasingly dilute mixtures using the same dilution ratio for each increasingly dilute mixture.

4. The method of claim 1 wherein the human genetic material is associated with a health effect in humans.

5. The method of claim 1 wherein the first mixture is serially diluted using an average dilution ratio of no more than approximately 1:5.

6. The method of claim 1 wherein the first mixture is serially diluted at least 3 times.

7. The method of claim 1 wherein the concentration of the human genetic material in the first formulation is no more than 1×10⁻³ w/w or v/v.

8. The method of claim 1 wherein the first mixture is serially diluted with water, alcohol, glycerin, lactose, and/or sucrose.

9. A method comprising:
mixing genetic material and a diluting agent to form a first mixture;
repeatedly diluting at least a portion of the first mixture to produce successive, increasingly dilute mixtures; and
succussing each increasingly dilute mixture;
wherein succussing each increasingly dilute mixture includes subjecting each increasingly dilute mixture to an impact force; and
wherein the genetic material is non-native genetic material.

10. The method of claim 9 wherein the same dilution ratio is used to produce each increasingly dilute mixture.

11. The method of claim 9 wherein the genetic material includes human genetic material.

12. The method of claim 9 wherein the genetic material is associated with a health effect in humans.

13. The method of claim 9 wherein each increasingly dilute mixture is diluted using an average dilution ratio of no more than approximately 1:5.

14. The method of claim 9 wherein repeatedly diluting at least a portion of the first mixture produces at least 3 increasingly dilute mixtures.

15. The method of claim 9 wherein the concentration of the genetic material in the last increasingly dilute mixture is no more than 1×10⁻³ w/w or v/v.

16. The method of claim 9 wherein each increasingly dilute mixture is diluted with water, alcohol, glycerin, lactose, and/or sucrose.

17. A method comprising:
obtaining a genetic sample from a patient;
identifying genetic material from the genetic sample that is associated with a health effect in the patient; and
orally administering a formulation prepared from the genetic material to the patient.

18. The method of claim 17 comprising:
mixing the genetic material and a diluting agent to form a first mixture; and
repeatedly diluting at least a portion of the first mixture to produce successive, increasingly dilute mixtures;
wherein the formulation includes one of the increasingly dilute mixtures.

19. The method of claim 17 wherein the genetic material includes an allele that is associated with a health effect in humans.

20. The method of claim 17 wherein the concentration of the genetic material in the formulation is no more than 1×10⁻³ w/w or v/v.

21. A formulation produced by any of the methods in claims 1 to 22.

22. The formulation of claim 21 comprising instructions for oral ingestion of the formulation by a human.

23. The formulation of claim 22 wherein the formulation is a homeopathic remedy.

24. A homeopathic remedy comprising:
a container;
a formulation inside the container;
a label attached to the container;
wherein the label communicates that the formulation includes non-native human genetic material at a potency of at least 1X.

25. The homeopathic remedy of claim 24 wherein the label communicates that the formulation includes non-native genetic material at a potency of at least 3X.

26. The homeopathic remedy of claim 24 wherein the label communicates that the formulation includes a risk allele associated with a human health effect.

27. The homeopathic remedy of claim 24 wherein the label communicates that the formulation is homeopathic in nature.

28. The homeopathic remedy of claim 24 wherein the formulation is liquid.

29. The homeopathic remedy of claim 24 wherein the formulation is in the form of a pellet, table, or capsule.
